(19) United States
(12) Patent Application Publication
(10) Pub. No.: US 2007/0025917 A1
(43) Pub. Date: Feb. 1, 2007

(54) Optical imaging based on locally activated bioluminescence

(75) Inventors: Peifang Tian, Niskayuna, NY (US); Robert John Filkins, Niskayuna, NY (US); Siavash Yazdanfar, Schenectady, NY (US); Stephen Johnson Lomnes, Albany, NY (US); Andrew John Healey, Moss (NO); Pavel Alexeyevich Fomitchov, New York, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/189,892

(22) Filed: Jul. 26, 2005

(51) Int. Cl. A61K 49/00; A61K 38/44

(52) U.S. Cl. 424/9.6; 424/94.4

(57) ABSTRACT

Methods and compositions for detecting and localizing light from a subject are disclosed. Also disclosed are methods for targeting light emission to selected regions, as well as for tracking entities within the subject. Also disclosed are methods for locating a target in a subject, such as by detecting light emission from a target site.

### BACKGROUND OF THE INVENTION

The invention relates to optical imaging. In particular, the invention relates to methods of locating a target in a subject based on localizing and detecting light emitting entities.

As shown in FIG. 1, it is known that luciferin, in the presence of the luciferase, combines with oxygen to generate light, i.e. bioluminescence. Other factors such as a suitable level of ATP, pH, and ion concentrations such as Ca²⁺ and Mg²⁺ are also often necessary to generate bioluminescence. The bioluminescence usually has a broad spectrum. A need exists to localize the bioluminescence to specific target, such as tumor, to thereby identify the target by the bioluminescense.

However, known methods of optical imaging have several disadvantages. For example, optical imaging either has limited penetration depth or resolution or both due to light scattering and absorption inside turbid media such as human bodies and biological tissues.

Consequently, a need still exists for improved optical imaging methods, specifically using ultrasonic activated bioluminescence to identify specific sites of interest.

### SUMMARY OF THE INVENTION

The purpose and advantages of embodiments of the invention will be set forth and apparent from the description that follows, as well as will be learned by practice of the embodiments of the invention. Additional advantages will be realized and attained by the methods and systems particularly pointed out in the written description and claims hereof, as well as from the appended drawings.

Bioluminescence to identify specific sites of interest are disclosed. Accordingly, one aspect of the invention includes a method of locating a target in a subject. The method includes (i) administering a vesicle having a first part of a light emitting entity to a subject; and administering a second part of a light emitting entity to the subject; (ii) allowing the vesicle or the second part of the light emitting entity to localize at a target; and (iii) applying an ultrasonic beam to the subject. The vesicle or the second part of a light emitting entity is conjugated to a targeting species that binds to a target or a marker substance produced by or associated with the target. The first part of the light emitting entity and the second part of the light emitting are capable of emitting light of a particular color when allowed to interact.

A second aspect of the invention includes a method of locating a target in a subject. The method includes (i) administering a vesicle to a subject and administering luciferin to the subject; (ii) allowing the vesicle to localize at a target; and (iii) applying an ultrasonic beam to the subject. The vesicle comprises luciferase and is conjugated to a targeting species that binds to a target or a marker substance produced by or associated with the target. The luciferase and the luciferin are capable of emitting light of a particular color when allowed to interact.

A third aspect of the invention includes a method of locating a target in a subject. The method includes (i) administering a vesicle to a subject and administering luciferase to the subject; (ii) allowing the vesicle to localize at a target; and iii) applying an ultrasonic beam to the subject. The vesicle comprises luciferin and is conjugated to a targeting species that bind to a target or a marker substance produced or associated with the target. The luciferase and the luciferin are capable of emitting light of a particular color when allowed to interact.

A fourth aspect of the invention includes a method of locating a target in a subject. The method includes (i) administering a vesicle comprising luciferase to a subject and administering luciferin conjugate to the subject; (ii) allowing the luciferin conjugate to concentrate at the target; and (iii) applying an ultrasonic beam to the subject. The luciferin conjugate is conjugated to a targeting species that binds to a target or a marker substance produced by or associated with the target. The luciferase and the luciferin are capable of emitting light of a particular color when allowed to interact.

A fifth aspect of the invention includes a method of locating a target in a subject. The method includes (i) administering a vesicle comprising luciferin to a subject and administering luciferase conjugate to the subject; (ii) allowing the luciferase conjugate to concentrate at the target; and iii) applying an ultrasonic beam to the subject. The luciferase conjugate is conjugated to a targeting species that bind to a target or a marker substance produced or associated with the target. The luciferase and the luciferin are capable of emitting light of a particular color when allowed to interreact

The accompanying figures, which are incorporated in and constitute part of this specification, are included to illustrate and provide a further understanding of the method and system of the invention. Together with the description, the figures serve to explain the principles of the invention.

### BRIEF DESCRIPTION OF THE DRAWINGS

FIG. 1 is a known schematic representation of luciferin, in the presence of the luciferase, combining with oxygen to generate light;

FIG. 2 is a schematic representation of luciferin and a vesicle comprising luciferase in accordance with an embodiment of the invention;

FIG. 3 is a schematic representation of luciferase and a vesicle comprising luciferin in accordance with an embodiment of the invention;

FIG. 4 is a schematic representation of luciferin conjugate and a vesicle comprising luciferase in accordance with an embodiment of the invention;

FIG. 5 is schematic representation of luciferase conjugate and a vesicle comprising luciferin in accordance with an embodiment of the invention; and

FIG. 6 is a schematic representation of a method of detecting a target site by administering luciferin and a vesicle comprising luciferase in accordance with an embodiment of the invention.

### DETAILED DESCRIPTION OF THE INVENTION

Reference will now be made in detail to exemplary embodiments of the invention, which are illustrated in the accompanying figures and examples. Referring to the drawings in general, it will be understood that the illustrations are for the purpose of describing a particular embodiment of the invention and are not intended to limit the invention thereto.

Whenever a particular embodiment of the invention is said to comprise or consist of at least one element of a group and combinations thereof, it is understood that the embodiment may comprise or consist of any of the elements of the group, either individually or in combination with any of the other elements of that group. Furthermore, when any variable occurs more than one time in any constituent or in formula, its definition on each occurrence is independent of its definition at every other occurrence. Also, combinations of substituents and/or variables are permissible only if such combinations result in stable compounds.

The ability to monitor the progression of various diseases is limited by the current methods of detecting the target site in diseased tissues. Experience may offer, in some cases, an estimate of probable sites and the progress of a disease. However, it is more often the case that the sites of the disease, and the pace of the disease are either not known or can only roughly be estimated. Moreover, because the progression of a disease is often individualized and unique, analyses of many disease targets need to be conducted to determine, on the average, what course a disease will follow.

An aspect of the invention provides methods of identifying specific target sites of interest, such as a tumor, by localizing ultrasonic activated bioluminescence to the target site.

A general method includes administering a vesicle having a first part of a light emitting entity to a subject; and administering a second part of a light emitting entity to the subject. The vesicle and the second part of the light emitting entity may be sequentially or simultaneously administered to the subject. The vesicle or the second part of a light emitting entity is conjugated to a targeting species that binds to a target or a marker substance produced by or associated with the target. The first part of the light emitting entity and the second part of the light emitting are capable of emitting light of a particular color when allowed to interact. In one embodiment, the two light emitting entities, luciferease and luciferein, interact as follows: Luciferase catalyzes the reaction of luciferin and molecular oxygen. This reaction generates some intermediate products, one of which is the electronic excited state of some chromophore which emits light. Luciferin and some light emitting intermediate products may bound onto luciferase.

The emitted light may have a broad spectral content, normally in the visible regime. In one embodiment, the full width half maximum width is about 100 nm.

The vesicle is any vesicle configured or functionalized to prevent the two parts of the light-emitting entities from interacting. The vesicle is functionalized or configured to contain or retain luciferase and or luciferin until disrupted or acted upon by a force to at least partially release the luciferase and or luciferin. In one embodiment, containing or retaining means containing or retaining a majority of the luciferase and or luciferin for at least about 2-3 hours. The vesicle may be of various shapes, such as elongated and circular, as well as various dimensions and material. In one embodiment, the vesicle is spherical and has a coating and core. In a particular embodiment, the vesicle includes a perfluorocarbon nano-emulsions core and a lipid coating. In another particular embodiment, the vesicle is a microsphere with a biodegradable polymeric coating. It should be appreciated that aspects of the invention includes adjusting the shape, size, and material of the vesicle to suit various needs such as allow access to a tumor site or minimize background noise based on detailed kinetics.

Next, the vesicle or the second part of a light emitting entity, whichever is conjugated to the targeting species, is allowed to concentrate at the target. An ultrasonic beam is then applied to the subject within the region of interest. The ultrasonic beam controls the interaction of the two light emitting entities by disrupting the vesicle, and thus at least partially releasing its contents, at or near the site of the target and allowing the two light emitting entities to interact. In a particular embodiment, the ultrasonic beam at least partially releases the first part of the light emitting entity from the vesicle, wherein the at least partially released first part of the light emitting entity interacts with the second part of the light emitting entity to emit a light of a particular color. In a particular embodiment, the emitted light is localized at the target. The bioluminescence is localized since it only comes from a target site. The focused ultrasonic beam activates the bioluminescence by allowing controlled released interaction of the parts of the light-emitting entities.

It should be appreciated that the method further comprises administering a plurality of the vesicles. It should also be appreciated that the vesicle at each occurrence are independent of the vesicle at every other occurrence, unless otherwise noted. For example, in one embodiment, the vesicles can be the same type, such as wherein two or more vesicles both comprise luciferase or both comprise luciferin. In another embodiment, the vesicles can be different, such as wherein one vesicle comprises luciferase and another vesicle comprises luciferin. Also, combinations of substituents and/or variables are permissible only if such combinations result in stable compounds. It should also be appreciated that administering includes any form of administration, such as but not limited to, orally, topically, parenterally, by inhalation spray or rectally. The term parenteral as used herein includes subcutaneous injections, intravenous, intramuscular, intrasternal injection or infusion techniques.

It should also be appreciated that the vesicles may have a plurality of the first part of a light emitting entity or a plurality of the second part of the light emitting entity. The plurality of the first part of the light emitting entity or the plurality of the second part of the light emitting entity is capable of emitting a plurality of wavelengths or lights when allowed to interact. The plurality of lights includes at least another color other the particular color already generated.

In addition to providing different light emitting entities, a plurality of colors may also be generated by adjusting or controlling the environmental factors of the vesicle. For example, environmental factors such as pH, concentration of ions like sodium, zinc, Ca, and Mg, and temperature, either individually or in combinations, affect the emission wavelength and hence the color generated. In fact, environmental factors such as pH and concentrations of ions can affect bioluminescence even to the point of whether there will be or will not be bioluminescence. Several factors affect the color of a bioluminescence. For example, in one case, the emission matches the fluorescence of an excited luciferase-bound product of the reaction (of luciferin and molecular oxygen). The luciferase structure can alter the color, as in a firefly, where single amino acid substitutions in the luciferase result in shifts in the emission spectrum. In bacteria and coelenterates, the chromophores of accessory proteins such as yellow fluorescent protein and green fluorescent protein (GFP) emit light, thus altering colors as well. Consequently, a system could be created such that upon disruption or release from the vesicle, the appropriate ionic microenvironment exists within the vesicle to discriminate the two reporters spectrally. Although the environment of the target may also affect the emission spectrum, one of ordinary skill in the art can make adjustments to counter the target environment. There are existing publications demonstrating a shift in emission wavelength as a function of various environmental factors.

A plurality of lights may also be generated by providing within the vesicle, in addition to the luciferin/luciferase, an energy acceptor that emits at a different wavelength than the luciferin. Thus, the energy released upon the release of the vesicle will excite the energy acceptor (e.g., a fluorescent protein) that can be detected by its fluorescence emission. This concept, similar to fluorescence resonance energy transfer (FRET), is sometimes referred to as bioluminescence resonance energy transfer (BRET), and has been demonstrated using luciferase as the energy donor and enhanced yellow fluorescent protein (EYFP) as the acceptor, and occurs naturally in certain species of Renilla that express green fluorescent protein (GFP). (Xu et al., Proc. Natl. Acad. Sci USA 96:151 (1999), Wilson and Hastings, Annu. Rev. Cell Dev. Biol. 14:197 (1998)).

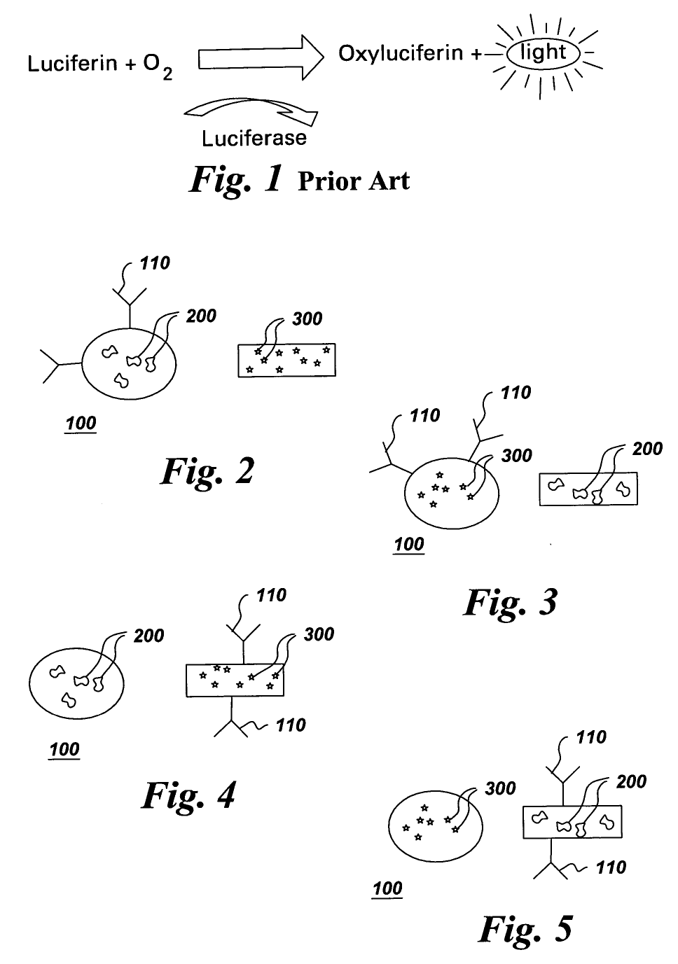

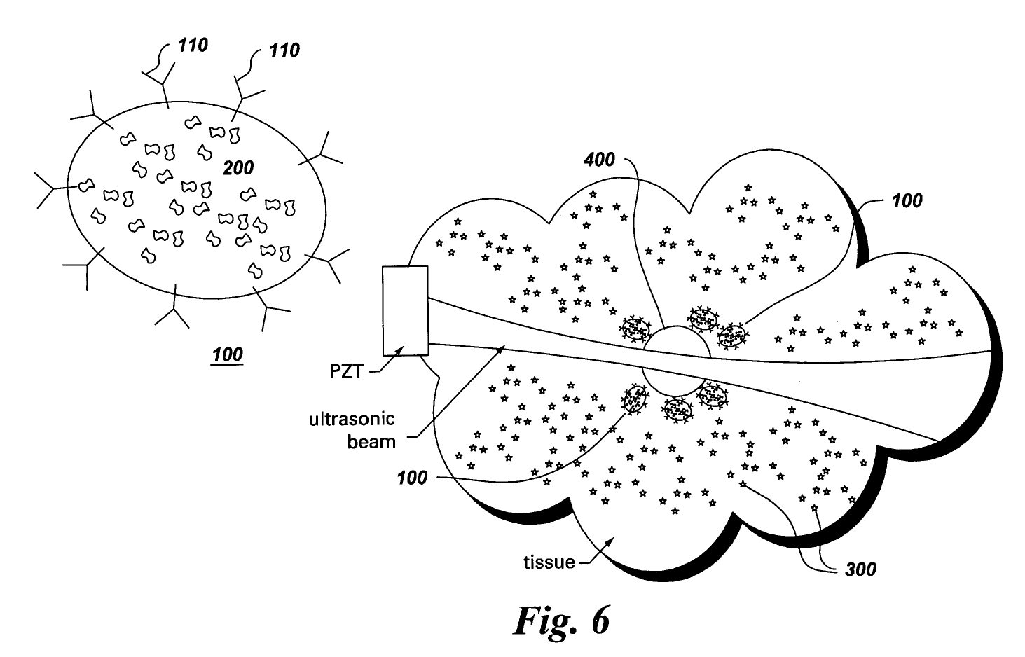

FIG. 2-5 and Table 1 disclose, but are not limited to, four particular embodiments of the general method described herein above. The four embodiments in FIG. 2-5 and Table 1 correspond to what the vesicle comprises, either luciferase (FIG. 2) or luciferin (FIG. 3), and to what the targeting species is conjugated. FIG. 6 is a schematic representation of a method of detecting a target site by administering luciferin and a vesicle comprising luciferase in accordance with an embodiment of the invention.

TABLE 1

| | | Targeting species conjugated |
|---|---|---|
| FIG. | vesicle includes | to |
| | luciferase | vesicle |
| | luciferin | vesicle |
| | luciferase | luciferin |
| | luciferin | luciferase |

As shown in FIG. 2 and FIG. 6, in one embodiment, the vesicle 100 comprises luciferase 200 and is conjugated to the targeting species 110 that binds to a target 400 or a marker substance produced by or associated with the target. In this embodiment of a method of locating a target in a subject, luciferin 300 and the vesicle 100 are administered to the subject. The luciferin and the vesicle comprising luciferase may be sequentially or simultaneously administered to the subject. The luciferin may also be administered in another vesicle. This another vesicle may also be conjugated to the targeting species 110 for further localization.

Next, the vesicle conjugated to the targeting species is allowed to concentrate at the target. In one embodiment, concentrate means a greater amount of the vesicle is found by the target than another non-target area. An ultrasonic beam is then applied to the subject. The luciferase and the luciferin are capable of emitting light of a particular color when allowed to interact. In one embodiment, the ultrasonic beam at least partially releases luciferase from the vesicle, wherein the at least partially released luciferase interacts with the luciferin to emit a light of a particular color. In a further embodiment, the emitted light is localized at the target.

It should be appreciated that the vesicle may be conjugated to a plurality of the targeting species 110. The method also further comprises administering to the subject a plurality of the vesicles. The plurality of the vesicles may comprise a plurality of luciferase. The plurality of luciferase interacts with the luciferin to emit a plurality of light. The plurality of lights includes at least another color other the particular color already generated. The plurality of lights may be used to detect multiple targets.

As shown in FIG. 3, in another embodiment, the vesicle 100 comprises luciferin 300 and is conjugated to the targeting species 110 that binds to a target 400 or a marker substance produced by or associated with the target. In this embodiment of a method of locating a target in a subject, luciferase 200 and the vesicle 100 are administered to the subject. The luciferase 200 and the vesicle comprising luciferin 300 may be sequentially or simultaneously administered to the subject. The luciferase may also be administered in another vesicle. This another vesicle may also be conjugated to the targeting species 110 for further localization.

Next, the vesicle conjugated to the targeting species is allowed to concentrate at the target. An ultrasonic beam is then applied to the subject. The luciferase and the luciferin are capable of emitting light of a particular color when allowed to interact. In one embodiment, the ultrasonic beam at least partially releases luciferin from the vesicle, wherein the at least partially released luciferin interacts with the luciferase to emit a light of a particular color. In a further embodiment, the emitted light is localized at the target.

It should be appreciated that the vesicle may be conjugated to a plurality of the targeting species 110. The method also further comprises administering to the subject a plurality of the vesicles. The plurality of the vesicles may comprise a plurality of luciferin. The plurality of luciferin interacts with the luciferase to emit a plurality of light. The plurality of lights includes at least another color other the particular color already generated.

As shown in FIG. 4-5, the vesicle 100 comprising luciferase 200 or luciferin 300 may be, but does not have to be, conjugated to the targeting species 110. As shown in FIG. 4, in one embodiment, luciferin 300 is conjugated to one or more targeting species 110 that binds to a target 400 or a marker substance produced by or associated with the target and the vesicle 100 comprises luciferase. The method comprises administering this luciferin conjugate and the vesicle comprising luciferase to a subject. The vesicle and the luciferin conjugate may be sequentially or simultaneously administered to the subject. The luciferin conjugate may be administered in another vesicle as well.

Next, the luciferin conjugated to the targeting species is allowed to concentrate at the target. An ultrasonic beam is then applied across the subject. The luciferase and the luciferin are capable of emitting light of a particular color when allowed to interact. In an embodiment, the ultrasonic beam at least partially releases luciferase from the vesicle. The at least partially released luciferase interacts with the luciferin to emit light of a particular color. In a further embodiment, the emitted light is localized at the target.

It should be appreciated that the vesicle may be conjugated to a plurality of the targeting species 110. The method further comprises administering to the subject a plurality of the vesicles. The plurality of the vesicles may comprise a plurality of luciferase. The plurality of luciferase interacts with the luciferin to emit a plurality of light. The plurality of lights includes at least another color other the particular color already generated.

As shown in FIG. 5, in another embodiment, luciferase 200 is conjugated to one or more targeting species that binds to a target 400 or a marker substance produced by or associated with the target and the vesicle comprises luciferin 300. The method comprises administering this luciferase conjugate and the vesicle comprising luciferin to a subject. The vesicle and the luciferase conjugate may be sequentially or simultaneously administered to the subject. The luciferase conjugate may be administered in another vesicle.

Next, the luciferase conjugated to the targeting species is allowed to concentrate at the target. An ultrasonic beam is then applied to the subject. The luciferase and the luciferin are capable of emitting light of a particular color when allowed to interact. In one embodiment, the ultrasonic beam at least partially releases luciferin from the vesicle, wherein the at least partially released luciferin interacts with the luciferase to emit light. The emitted light is localized at the target.

It should be appreciated that the vesicle may be also conjugated to a plurality of the targeting species 110. The method further comprises administering to the subject a plurality of the luciferase. The plurality of luciferase interacts with the luciferin to emit a plurality of light. The plurality of lights includes at least another color other the particular color already generated.

Luciferase

Luciferase and luciferin includes any enzyme and substrate in a bioluminescent reaction. Usually, luciferase and luciferin are specified by the organism, such as bacterial, marine, firefly, and dinoflagellate luciferase and luciferin. Particular examples include marine luciferase and luciferin. More particular examples include Gaussia, Oplophorus, Vargula, coelenterate, and ctenophore.

Luciferin

In a particular embodiment, luciferin includes: 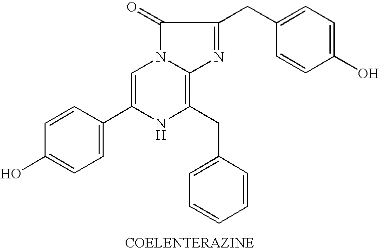
as disclosed in Bioluminescence, Wilson and Hastings Annu. Rev. Cell. Dev. Biol. 14, 197-230 (1998).

In another particular embodiment, luciferin includes the firefly luciferin: 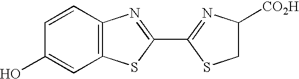
and derivatives and analogs thereof.
Ultrasound Beam

It should be appreciated that application of the ultrasonic beam to the subject encompasses a wide range of factors, such as frequency, duration, acoustic pressure, how generated, and how applied to the subject.

Examples of frequency range include, but are not limited to, from about 0.05 MHz to about 100 MHz, particularly about 0.5-10 MHz.

Examples of duration include, but are not limited to, 0.05-10 us for single ultrasonic pulses and pulse trains, 1-20 cycles tone bursts, and continuous wave (CW) sinusoidal ultrasonic generation.

Examples of acoustic pressure include, but are not limited to, up to about 2.0 MPa., particularly up to about 1.5 Mpa for a human body subject.

Examples of generating the ultrasound include, but are not limited to, the following ways. The ultrasound can be generated by a single transducer that is scanned across the subject or ultrasound can be generated and scanned across the subject by a array of transducers such as phased arrays. Ultrasound can be generated by piezo-electric transducers, and opto(photo)-acoustic sources. Ultrasonic sources can be applied outside of the subject or internally using an embedded catheter-like probe.

Targeting Species

As shown in FIG. 2-5, the targeting species 110 has one or more targeting moieties that bind to a target site 400 or to a substance produced by or associated with the target site via a primary binding site. Furthermore, as shown in FIG. 2, the targeting species may bind to one or more targets 400 or a biomarker produced by or associated with the in-vivo-target. The target site is a specific site, such as a cell or group of cells, tissue, organ, tumor, or lesion. The targeting moiety binds to the target site or to a substance produced by or associated with the target site via a primary binding site. Non-limiting examples of targeting species include, but are not limited to, proteins, peptides, polypeptides, glycoproteins, lipoproteins, phospholipids, oligonucleotides, steroids, alkaloids or the like, e.g., hormones, lymphokines, growth factors, albumin, cytokines, enzymes, immune modulators, receptor proteins, antisense oligonucleotides, antibodies and antibody fragments, which preferentially bind marker substances that are produced by or associated with the target site.

Proteins are known that preferentially bind marker substances that are produced by or associated with lesions. For example, antibodies can be used against cancer-associated substances, as well as against any pathological lesion that shows an increased or unique antigenic marker, such as against substances associated with cardiovascular lesions, for example, vascular clots including thrombi and emboli, myocardial infarctions and other organ infarcts, and atherosclerotic plaques; inflammatory lesions; and infectious and parasitic agents.

Cancer states include carcinomas, melanomas, sarcomas, neuroblastomas, leukemias, lymphomas, gliomas, myelomas, and neural tumors.

Infectious diseases include those caused by invading microbes or parasites. As used herein, “microbe” denotes virus, bacteria, rickettsia, mycoplasma, protozoa, fungi and like microorganisms, “parasite” denotes infectious, generally microscopic or very small multicellular invertebrates, or ova or juvenile forms thereof, which are susceptible to antibody-induced clearance or lytic or phagocytic destruction, e.g., malarial parasites, spirochetes and the like, including helminths, while “infectious agent” or “pathogen” denotes both microbes and parasites.

The protein substances useful as targeting species in the invention include protein, peptide, polypeptide, glycoprotein, lipoprotein, or the like; e.g. hormones, lymphokines, growth factors, albumin, cytokines, enzymes, immune modulators, receptor proteins, antibodies and antibody fragments.

The protein substances of particular interest are antibodies and antibody fragments. The terms “antibodies” and “antibody fragments” mean generally immunoglobulins or fragments thereof that specifically bind to antigens to form immune complexes.

The antibody may be a whole immunoglobulin of any class; e.g., IgG, IgM, IgA, IgD, IgE, chimeric or hybrid antibodies with dual or multiple antigen or epitope specificities. It can be a polyclonal antibody, particularly a humanized or an affinity-purified antibody from a human. It can be an antibody from an appropriate animal; e.g., a primate, goat, rabbit, mouse, or the like. If the target site-binding region is obtained from a non-human species, the target species may be humanized to reduce immunogenicity of the non-human antibodies, for use in human diagnostic or therapeutic applications. Such a humanized antibody or fragment thereof is also termed “chimeric.” For example, a chimeric antibody comprises non-human (such as murine) variable regions and human constant regions. A chimeric antibody fragment can comprise a variable binding sequence or complementarity-determining regions (“CDR”) derived from a non-human antibody within a human variable region framework domain. Monoclonal antibodies are also suitable because of their high specificities. Monoclonal antibodies are readily prepared by what are now considered conventional procedures of immunization of mammals with an immunogenic antigen preparation, fusion of immune lymph or spleen cells with an immortal myeloma cell line, and isolation of specific hybridoma clones. More unconventional methods of preparing monoclonal antibodies are also included, such as interspecies fusions and genetic engineering manipulations of hypervariable regions, since it is primarily the antigen specificity of the antibodies that affects their utility. It will be appreciated that newer techniques for production of monoclonal antibodies (“MAb”) can also be used; e.g., human MAbs, interspecies MAbs, chimeric (e.g., human/mouse) MAbs, genetically engineered antibodies, and the like.

Useful antibody fragments include F(ab′)₂, F(ab)₂, Fab′, Fab, Fv, and the like including hybrid fragments. Particular fragments are Fab′, F(ab′)₂, Fab, and F(ab)₂. Also useful are any subfragments retaining the hypervariable, antigen-binding region of an immunoglobulin and having a size similar to or smaller than a Fab′ fragment. An antibody fragment can include genetically engineered and/or recombinant proteins, whether single-chain or multiple-chain, which incorporate an antigen-binding site and otherwise function in vivo as targeting species in substantially the same way as natural immunoglobulin fragments. Such single-chain binding molecules are disclosed in U.S. Pat. No. 4,946,778. Fab′ antibody fragments may be conveniently made by reductive cleavage of F(ab′)₂ fragments, which themselves may be made by pepsin digestion of intact immunoglobulin. Fab antibody fragments may be made by papain digestion of intact immunoglobulin, under reducing conditions, or by cleavage of F(ab)₂ fragments which result from careful papain digestion of whole immunoglobulin. The fragments may also be produced by genetic engineering.

It should be noted that mixtures of antibodies and immunoglobulin classes can be used, as can hybrid antibodies. Multispecific, including bispecific and hybrid, antibodies and antibody fragments are sometimes desirable for detecting and treating lesions and comprise at least two different substantially monospecific antibodies or antibody fragments, wherein at least two of the antibodies or antibody fragments specifically bind to at least two different antigens produced or associated with the targeted lesion or at least two different epitopes or molecules of a marker substance produced or associated with the targeted lesion. Multispecific antibodies and antibody fragments with dual specificities can be prepared analogously to the anti-tumor marker hybrids disclosed in U.S. Pat. No. 4,361,544. Other techniques for preparing hybrid antibodies are disclosed in; e.g., U.S. Pat. Nos. 4,474,893 and 4,479,895, and in Milstein et al., Immunology Today, Vol. 5, 299 (1984).

Particular proteins that may be used are proteins having a specific immunoreactivity to a biomarker substance of at least 60% and a cross-reactivity to other antigens or non-targeted substances of less than 35%.

As disclosed above, antibodies against tumor antigens and against pathogens are known. For example, antibodies and antibody fragments which specifically bind biomarkers produced by or associated with tumors or infectious lesions, including viral, bacterial, fungal and parasitic infections, and antigens and products associated with such microorganisms have been disclosed in Hansen et al. (U.S. Pat. No. 3,927,193) and Goldenberg (U.S. Pat. Nos. 4,331,647, 4,348,376, 4,361,544, 4,468,457, 4,444,744, 4,818,709 and 4,624,846). In particular, antibodies against an antigen, e.g., a gastrointestinal, lung, breast, prostate, ovarian, testicular, brain or lymphatic tumor, a sarcoma, or a melanoma, are advantageously used.

A wide variety of monoclonal antibodies against infectious disease agents have been developed, and are summarized in a review by Polin, in Eur. J. Clin. Microbiol., 3(5):387-398, 1984. These include MAbs against pathogens and their antigens. Exemplary infectious disease agents are disclosed in U.S. Pat. No. 5,482,698.

Additional examples of MAbs generated against infectious organisms that have been described in the literature are noted below.

MAbs against the gp 120 glycoprotein antigen of human immunodeficiency virus 1 (HIV-1) are known, and certain of such antibodies can have an immunoprotective role in humans. See, e.g., Rossi et al., Proc. Natl. Acad. Sci. USA, Vol. 86, pp. 8055-58 (1990). Other MAbs against viral antigens and viral-induced antigens are also known. MAbs against malaria parasites can be directed against the sporozoite, merozoite, schizont and gametocyte stages.

Suitable MAbs have been developed against most of the microorganisms (bacteria, viruses, protozoa, other parasites) responsible for the majority of infections in humans, and many have been used previously for in vitro diagnostic purposes. These antibodies, and newer MAbs that can be generated by conventional methods, are also appropriate for use.

Proteins useful for detecting and/or treating cardiovascular lesions include fibrin-specific proteins; for example, fibrinogen, soluble fibrin, antifibrin antibodies and fragments, fragment E₁ (a 60 kDa fragment of human fibrin made by controlled plasmin digestion of crosslinked fibrin), plasmin (an enzyme in the blood responsible for the dissolution of fresh thrombi), plasminogen activators (e.g., urokinase, streptokinase and tissue plasminogen activator), heparin, and fibronectin (an adhesive plasma glycoprotein of 450 kDa) and platelet-directed proteins; for example, platelets, antiplatelet antibodies, and antibody fragments, anti-activated platelet antibodies, and anti-activated platelet factors, which have been reviewed by Koblik et al., Semin. Nucl. Med., Vol. 19, 221-237 (1989).

In one embodiment, the targeting species is an MAb or a fragment thereof that recognizes and binds to a heptapeptide of the amino terminus of the β-chain of fibrin monomer. Fibrin monomers are produced when thrombin cleaves two pairs of small peptides from fibrinogen. Fibrin monomers spontaneously aggregate into an insoluble gel, which is further stabilized to produce blood clots.

In another embodiment, the targeting species is a chimeric antibody derived from an antibody designated as NR-LU-10. This chimeric antibody has been designated as NR-LU-13 and disclosed in U.S. Pat. No. 6,358,710. NR-LU-13 contains the murine Fv region of NR-LU-10 and therefore comprises the same binding specificity as NR-LU-10. The chimeric antibody also comprises human constant regions. Thus, this chimeric antibody binds the NR-LU-10 antigen and is less immunogenic because it is made more human-like. NR-LU-10 is a nominal 150 kilodalton (or kDa) murine IgG_{2b} pan carcinoma monoclonal antibody that recognizes an approximately 40 kDa glycoprotein antigen expressed on most carcinomas, such as small cell lung, non-small cell lung, colon, breast, renal, ovarian, pancreatic, and other carcinoma tissues. The NR-LU-10 antigen has been further described by Varki et al., “Antigens Associated With a Human Lung Adenocarcinoma Defined by Monoclonal Antibodies,” Cancer Research, Vol. 44, 681-87 (1984), and Okabe et al., ““Monoclonal Antibodies to Surface Antigens of Small Cell carcinoma of the Lungs,” Cancer Research Vol. 44, 5273-78 (1984). Methods for preparing antibodies that binds to epitopes of the NR-LU-10 antigen are known and are disclosed in U.S. Pat. No. 5,084,396. One suitable method for producing monoclonal antibodies is the standard hybridoma production and screening process, which is well known in the art. In a particular embodiment, the targeting species is a humanized antibody or humanized antibody fragment that binds specifically to the antigen bound by antibody NR-LU-13. A humanization method comprises grafting only non-human CDRs onto human framework and constant regions (see; e.g., Jones et al., Nature, Volume 321, 522-35 (1986)). Another humanization method comprises transplanting the entire non-human variable domains, but cloaking (or veneering) these domains by replacement of exposed residues reduce immunogenicity (see; e.g., Padlan, Molec. Immun., Vol. 28, 489-98 (1991)). Exemplary humanized light and heavy sequences derived from the light and heavy sequences of the NR-LU-13 antibody are disclosed in U.S. Pat. No. 6,358,710, and are denoted therein as NRX451. The phrase “binds specifically” with respect to antibody or antibody fragment means such antibody or antibody fragment has a binding affinity of at least about 10⁴ M⁻¹. Particularly, the binding affinity is at least about 10⁶ M⁻¹, and more particularly, at least about 10⁸ M⁻¹.

According to still another embodiment, the targeting species is a humanized anti-p185^{HER2} antibody that specifically recognizes the p185 HER2 protein expressed on breast cancer cells. A humanized anti-p185^{HER2} antibody known as Herceptin is widely available. An anti-HER2 murine MAb known as ID5 is available from Applied BioTechnology/Oncogene Science (Cambridge, Mass.), which can be humanized according to conventional methods. See, e.g., X. F. Lee et al., “Differential Signaling by an Anti-p185^{HER2} Antibody and Hergulin,” Cancer Research, Vol. 60, 3522-31 (2000).

In other embodiments, the targeting species is an antibody or a fragment thereof, particularly a human or humanized antibody or fragment thereof, that is raised against one of anti-carcinogembryonic antigen (“CEA”), anti-colon-specific antigen-p (“CSAp”), and other well known tumor-associated antigens, such as CD19, CD 20, CD21, CD22, CD23, CD30, CD74, CD80, HLA-DR, I, MUC 1, MUC 2, MUC 3, MUC 4, EGFR, HER2/neu, PAM-4, Bre3, TAG-72 (C72.3, CC49), EGP-1 (e.g., RS7), EGP-2 (e.g., 17-1A and other Ep-CAM targets), Le(y (e.g., B3), A3, KS-1, S100, IL-2, T101, necrosis antigens, folate receptors, angiogenesis markers (e.g., VEGFR), tenascin, PSMA, PSA, tumor-associated cytokines, MAGE and/or fragments thereof. Tissue-specific antibodies (e.g., against bone marrow cells, such as CD34, CD74, etc., parathyroglobulin antibodies, etc.) as well as antibodies against non-malignant diseased biomarkers, such as macrophage antigens of atherosclerotic plaques (e.g., CD74 antibodies), and also specific pathogen antibodies (e.g., against bacteria, viruses, and parasites) are well known in the art.

It should be understood that the foregoing disclosure of various antigens or biomarkers that can be used to raise specific antibodies against them (and from which antibodies fragments may be prepared) serves only as examples, and is not to be construed in any way as a limitation of the invention.

The method of locating a target in a subject also further includes obtaining one or more base-line image of a portion of a subject suspected of having a disease condition before administering the luciferase and luciferin pair as disclosed in FIG. 2-5. Image as used herein includes signals, as well as any visual representation of the spatial distribution (or location) of an object. In one embodiment, an image consists of an array (of more than one dimension), where the values of the array typically represent an intensity associated with a spatial coordinate in two or three dimensions.

The method also includes comparing the base-line image with an additional image to evaluate the disease condition. The step of obtaining additional images to evaluate the disease condition may be repeated at different time intervals as desired. Thus, it should be appreciated that one or more base line images may be compared with one or more additional images or the additional images may be compared with each other to monitor the disease condition.

Another aspect of the invention provides a method for assessing an effectiveness of a prescribed regimen for treating a disease condition that is characterized by an overproduction or underproduction of a disease-specific substance or biomarker. The method includes obtaining a base-line image of a portion of a subject suspected of having the disease condition before administering the luciferase and luciferin pair as disclosed in FIG. 2-5. The luciferase and luciferin pair as disclosed in FIG. 2-5 are administered. The ultrasonic beam is applied to the subject. The method then includes obtaining a pre-treatment image coming from the same portion of the subject and treating the disease condition in the subject with a prescribed regimen. A post-treatment image coming from the same portion of the subject is then obtained.

The method may further comprise comparing the post-treatment image to the pre-treatment image to assess the effectiveness of the prescribed regimen, wherein a change in image contrast during a course of the prescribed regimen indicates that the treatment has provided benefit. The method may also further comprise comparing the post-treatment image to the baseline image to assess the effectiveness of the prescribed regimen, wherein a change in image contrast or signals during a course of the prescribed regimen indicates that the treatment has provided benefit. The method may also further comprises repeating the treatment and images steps at predetermined time intervals during the course of treating the disease condition. The ultrasonic beam is applied to the subject at different intervals as necessary to obtain images.

In various aspects of the methods, any one of the luciferase and luciferin pair that are disclosed in FIG. 2-5 and Table 1 may be administered to suit the particular circumstances and disease.

During the course of the treatment of the disease, a change in signal obtained from the imaging technique (compared to a base-line signal obtained before the treatment) of, for example, 10 percent or more can signify that the treatment has conferred some benefit. In another embodiment, a change in signal obtained from the imaging technique (compared to a base-line signal obtained before the treatment) of, for example, 20 percent or more can signify that the treatment has conferred some benefit. The prescribed regimen for treating the disease can be, for example, treatment with drugs, radiation, or surgery.

In one aspect, the invention provides a kit that comprises luciferase and luciferin pair as disclosed in FIG. 2-5 before use which are kept separate from each other.

### EXAMPLES

There are four ways to implement single color ultrasonic activated bioluminescence imaging as respectively disclosed in FIG. 2-5

FIG. 2 and FIG. 3:

### Example 1

In one embodiment, the vesicle comprising luciferase or luciferin is a microsphere with a biodegradable polymeric coating.

Since luciferases and many luciferins are soluble in aqueous solutions, techniques developed for hydrophilic drug-filled microspheres can be used to make a microsphere that contains or retains luciferase or luciferin. The luciferase or luciferin containing microsphere may be made of or made from biodegradable polymers such as polylactide, polylactidecoglyolide, and polycaprolactone. Techniques for making such luciferase or luciferin containing biodegradable polymers microsphere include double or multiple emulsion, phase separation (coacervation), spray drying. (Dissertation, Silke Mohl, Ludwig-Maximilians-Universität München). To make the microsphere responsive to ultrasound, a gas such as perfluorocarbon is enclosed in the microsphere. A perfluorocarbon gas such as perfluoropropane (C₃F₈) and perfluorobutane (C₄F₁₀)) is enclosed within microspheres by lyophilizing the microspheres and instilling gas into a headspace of a vial containing the microspheres. (Unger et al., “Therapeutic applications of lipid-coated microspheres”, Advanced Drug Delivery Reviews 56, 1291-1314 (2004)). In one embodiment, the average diameter of such a microsphere is around 1 micron.

### Example 2

In another embodiment, the vesicle comprising luciferase or luciferin has a perfluorocarbon nano-emulsions core and a lipid coating.

The core includes liquid perfluorocarbon (such as perfluoropentane, which is a liquid at room temperature, but which boils at about 29.5 degree C. The nano-emulsion vesicle can be prepared by incubating the perfluoropentane and lipids and either luciferase or luciferin. This liquid perfluorocarbon core is liquid at room temperature. However, the liquid perfluorocarbon core may undergo the phase transition from liquid to gaseous states at a suitable temperature. Insonation with ultrasound energy can also be used to stimulate the transition from liquid to gas, therefore, the vesicle becomes a gas vesicle. In one embodiment, the average size of this vesicle is about 200 nm.

The vesicle is then targeted at specific receptors by conjugating a targeting species to the vesicle.

The vesicle may be conjugated to the targeting species in various ways, such as attach receptor ligands, including monoclonal antibodies, polysaccharides and peptides that recognize disease antigens onto the microsphere surface. Binding, directly or via a flexible spacer arm, to the microsphere can involve covalent or non-covalent (hydrophobic, avidin-biotin pairing) interactions.

The vesicle is administered, such by injecting, into the subject; then gets concentrated onto target; the other component (i.e. the second light emitting entity) of bioluminescence (either luciferin or luciferase) is administered into the subject. In one embodiment, the second light emitting entity, which is not conjugated to a targeting species, is administered in excess. In another embodiment, the second light emitting entity distributes evenly in the subject. The subject is then scanned with an ultrasonic beam. Bioluminescence is emitted when the ultrasonic beam disrupts the vesicle such that its enclosed content (either luciferase or luciferin) leaks out. This happens when the focal region of the ultrasonic beam hits the vesicle, which is concentrated onto the target. Therefore, the bioluminescence is localized and reflects the position of the target.

When applying the ultrasonic beam, it has been shown that the pressure threshold for fragmentation (disruption) of a lipid-shelled microsphere increases with bubble size, acoustic frequency, and decreases with ultrasound pulse duration. (Chomas, Dayton, May, Ferrara, “Threshold of fragmentation for ultrasonic contrast agents”, J. Biomed. Opt. 6, 141-150 (2001)) For example, ultrasonic pulses of 1.6 MPa at 1.5 MHz can cause fragmentation of acoustically active lipospheres 3 μm in radius. (Shortencarier et al., “A method for radiation-force localized drug delivery using gas-filled lipospheres”, IEEE Trans. Ultrasonics, Ferroelectrics, and frequency control 51, 822-831, (2004)). Another particular example is three ultrasonic pulses of 5 cycles each at 1.5 MHz and 2 MPa, separated by 20 microseconds. The focal area is about 1 mm². It should be appreciated that the ultrasonic parameters may be adjusted to specific microspheres with different shell materials, thickness, and bubble size.

FIG. 4 and FIG. 5

In FIG. 4 and FIG. 5, the vesicle which includes one part of the light emitting entity is not conjugated to a targeting species. Instead, the other part of the bioluminescence light emitting entity is conjugated to a targeting species to concentrate at a target of choice. When the vesicle and the conjugated luciferin or luciferase are injected or delivered into the subject, the conjugated part will concentrate in the target while the vesicle will distribute evenly throughout the subject. When the ultrasonic beam scans through the subject, bioluminescence occurs when the focal region of ultrasonic beam disrupts the vesicle and when the vesicle is close to a conjugate. This method of concentrating by a target may be helpful when it is undesirable or difficult to conjugate the vesicle to the targeting species. In addition, it is also useful to drug discovery. For example, in FIG. 5, cells, infectious agents or genes can be labeled with luciferase or its gene, and a specific property of the cells or agents (for example, their numbers) can then be monitored as a function of time. This is widely used in bioluminescence imaging. Combined with microspheres and ultrasonic excitation, the location of the specific cells can be determined more accurately.

While the invention has been described in detail in connection with only a limited number of aspects, it should be readily understood that the invention is not limited to such disclosed aspects. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A method of locating a target in a subject comprising:
(i) administering a vesicle comprising a first part of a light emitting entity to a subject; and administering a second part of a light emitting entity to the subject;
wherein the vesicle or the second part of a light emitting entity is conjugated to a targeting species that binds to a target or a marker substance produced by or associated with the target;
(ii) allowing the vesicle or the second part of a light emitting entity conjugated to the targeting species to concentrate at the target; and
(iii) applying an ultrasonic beam to the subject.

2. The method of claim 1, wherein the ultrasonic beam at least partially releases the first part of the light emitting entity from the vesicle, wherein the at least partially released first part of the light emitting entity interacts with the second part of the light emitting entity to emit light of a particular color.

3. The method of claim 2, wherein the emitted light is localized at the target.

4. The method of claim 1, wherein the vesicle and the second part of the light emitting entity are sequentially administered to the subject.

5. The method of claim 1, wherein the vesicle and the second part of the light emitting entity are simultaneously administered to the subject.

6. The method of claim 1, wherein the vesicle is conjugated to the targeting species that binds to a target or a marker substance produced by or associated with the target and wherein the first part of the light emitting entity is luciferase.

7. The method of claim 1, wherein the vesicle is conjugated to the targeting species that binds to a target or a marker substance produced by or associated with the target and wherein the first part of the light emitting entity is luciferin.

8. The method of claim 1, wherein the second part of the light emitting entity is conjugated to the targeting species that binds to a target or a marker substance produced by or associated with the target and wherein the second part of the light emitting entity is luciferin.

9. The method of claim 1, wherein the second part of the light emitting entity is conjugated to the targeting species that binds to a target or a marker substance produced by or associated with the target and wherein the second part of the light emitting entity is luciferase.

10. The method of claim 1, wherein administering the second part of the light emitting entity comprises administering the second part of the light emitting entity in another vesicle.

11. The method of claim 1, further comprising administering to the subject a plurality of the vesicles comprising a plurality of the first part of a light emitting entity or a plurality of the second part of the light emitting entity;
wherein the plurality of the first part of the light emitting entity or the plurality of the second part of a light emitting entity is capable of emitting a plurality of light when allowed to interact.
wherein the plurality of light is of another color than the particular color.

12. The method of claim 1, wherein the vesicle further comprises an energy acceptor.

13. The method of claim 1, further comprising controlling an environment of the vesicle by adjusting at least one factor selected from a group consisting of temperature, pH, and ions.

14. The method of claim 1, further comprising taking a baseline image of a portion of the subject before administering the vesicle and the second light emitting entity and taking a post image of the same portion of the subject after administering the vesicle and the second light emitting entity and comparing the baseline image with the post image.

15. A method of locating a target in a subject comprising:
(i) administering a vesicle to a subject and administering luciferin to the subject;
wherein the vesicle comprises luciferase and is conjugated to a targeting species that binds to a target or a marker substance produced by or associated with the target; and
(ii) allowing the vesicle conjugated to the targeting species to concentrate at the target; and
(iii) applying an ultrasonic beam to the subject.

16. The method of claim 15, wherein the ultrasonic beam at least partially releases luciferase from the vesicle, wherein the at least partially released luciferase interacts with the luciferin to emit light of a particular color.

17. The method of claim 16, wherein the emitted light is localized at the target.

18. The method of claim 15, wherein the luciferin and the vesicle comprising luciferase are sequentially administered to the subject.

19. The method of claim 15, wherein the luciferin and the vesicle comprising luciferase are simultaneously administered to the subject.

20. The method of claim 15, wherein the vesicle is conjugated to a plurality of targeting species.

21. The method of claim 15, further comprising administering to the subject a plurality of the vesicles comprising a plurality of luciferase;
wherein the plurality of luciferase interacts with the luciferin to emit a plurality of light;
wherein the plurality of light is of another color than the particular color.

22. The method of claim 15, wherein the vesicle further comprises an energy acceptor.

23. The method of claim 15, further comprising controlling an environment of the vesicle by adjusting at least one factor selected from a group consisting of temperature, pH, and ions.

24. The method of claim 15, further comprising taking a baseline image of a portion of the subject before administering the vesicle and the luciferin and taking a post image of the same portion of the subject after administering the vesicle and the luciferin and comparing the baseline image with the post image.

25. The method of claim 15, wherein administering the luciferin comprises administering the luciferin in another vesicle.

26. A method of locating a target in a subject comprising:
(i) administering a vesicle to a subject and administering luciferase to the subject;
wherein the vesicle comprises luciferin and is conjugated to a targeting species that bind to a target or a marker substance produced or associated with the target;
(ii) allowing the vesicle conjugated to the targeting species to concentrate at the target; and
(iii) applying an ultrasonic beam to the subject.

27. The method of claim 26, wherein the ultrasonic beam at least partially releases luciferin from the vesicle, wherein the at least partially released luciferin interacts with the luciferase to emit light.

28. The method of claim 27, wherein the emitted light is localized at the target.

29. The method of claim 26, wherein the luciferase and the vesicle comprising luciferin are sequentially administered to the subject.

30. The method of claim 26, wherein the luciferase and the vesicle comprising luciferin are simultaneously administered to the subject.

31. The method of claim 26, wherein the vesicle is conjugated to a plurality of targeting species.

32. The method of claim 26, further comprising administering to the subject a plurality of the luciferase;
wherein the plurality of luciferase interacts with the luciferin to emit a plurality of light;
wherein the plurality of light is another color than the particular color.

33. The method of claim 26, wherein the vesicle further comprises an energy acceptor.

34. The method of claim 26, further comprising controlling an environment of the vesicle by adjusting at least one factor selected from a group consisting of temperature, pH, and ions.

35. The method of claim 26, further comprising taking a baseline image of a portion of the subject before administering the vesicle and the luciferase and taking a post image of the same portion of the subject after administering the vesicle and the luciferase and comparing the baseline image with the post image.

36. The method of claim 26, wherein administering the luciferase comprises administering the luciferase in another vesicle.

37. The method of claim 36, wherein the another vesicle is conjugated to a targeting species.

38. A method of locating a target in a subject comprising:
(i) administering a vesicle comprising luciferase to a subject and administering luciferin conjugate to the subject;
wherein the luciferin conjugate is conjugated to a targeting species that binds to a target or a marker substance produced by or associated with the target; and
(ii) allowing the luciferin conjugated to the targeting species to concentrate at the target; and
(iii) applying an ultrasonic beam across the subject.

39. The method of claim 38, wherein the ultrasonic beam at least partially releases luciferase from the vesicle, wherein the at least partially released luciferase interacts with the luciferin to emit light.

40. The method of claim 39, wherein the emitted light is localized at the target.

41. The method of claim 38, wherein the vesicle and the luciferin conjugate are sequentially administered to the subject.

42. The method of claim 38, wherein the vesicle and the luciferin conjugate are simultaneously administered to the subject.

43. The method of claim 38, wherein the vesicle is conjugated to a plurality of targeting species.

44. The method of claim 38, wherein administering the luciferin conjugate comprises administering the luciferin conjugate in another vesicle.

45. The method of claim 38, further comprising administering to the subject a plurality of the vesicles;
wherein the plurality of the vesicles comprise a plurality of luciferase
wherein the plurality of luciferase interact with the luciferin to emit a plurality of light; and
wherein the plurality of light is of another color than the particular color.

46. The method of claim 38, wherein the vesicle further comprises an energy acceptor.

47. The method of claim 38, further comprising controlling an environment of the vesicle by adjusting at least one factor selected from a group consisting of temperature, pH, and ions.

48. The method of claim 38, further comprising taking a baseline image of a portion of the subject before administering the vesicle and the luciferin conjugate and taking a post image of the same portion of the subject after administering the vesicle and the luciferin conjugate and comparing the baseline image with the post image.

49. A method of locating a target in a subject comprising
(i) administering a vesicle comprising luciferin to a subject and administering luciferase conjugate to the subject;
wherein the luciferase conjugate is conjugated to a targeting species that bind to a target or a marker substance produced or associated with the target;
(ii) allowing the luciferase conjugated to the targeting species to concentrate at the target; and
(iii) applying an ultrasonic beam across the subject.

50. The method of claim 49, wherein the ultrasonic beam at least partially releases luciferin from the vesicle, wherein the at least partially released luciferin interacts with the luciferase to emit light.

51. The method of claim 50, wherein the emitted light is localized at the target.

52. The method of claim 49, wherein the vesicle and the luciferase conjugate are sequentially administered to the subject.

53. The method of claim 49, wherein the vesicle and the luciferase conjugate are simultaneously administered to the subject.

54. The method of claim 49, wherein the vesicle is conjugated to a plurality of targeting species.

55. The method of claim 49, further comprising administering to the subject a plurality of the luciferase;
wherein the plurality of luciferase interact with the luciferin to emit a plurality of light;
wherein the plurality of light is of another color than the particular color.

56. The method of claim 49, wherein the vesicle further comprises an energy acceptor.

57. The method of claim 49, further comprising controlling an environment of the vesicle by adjusting at least one factor selected from a group consisting of temperature, pH, and ions.

58. The method of claim 49, further comprising taking a baseline image of a portion of the subject before administering the vesicle and the luciferase conjugate and taking a post image of the same portion of the subject after administering the vesicle and the luciferase conjugate and comparing the baseline image with the post image.

59. The method of claim 49, wherein administering the luciferase conjugate comprises administering the luciferase conjugate in another vesicle.
